(19) United States
(12) Patent Application Publication
(10) Pub. No.: US 2005/0163872 A1
(43) Pub. Date: Jul. 28, 2005

(54) Compositions and methods for reducing cholesterol

(75) Inventor: Anil B. Khare, Crystal, MN (US)

Correspondence Address: CARGILL, INCORPORATED, LAW/24, 15407 MCGINTY ROAD WEST, WAYZATA, MN, 55391, US

(73) Assignee: CARGILL, INCORPORATED, Wayzata, MN (US)

(21) Appl. No.: 10/763,474

(22) Filed: Jan. 23, 2004

(51) Int. Cl. A61K 35/78; A61K 31/715; A61K 31/56

(52) U.S. Cl. 424/748; 514/54; 514/171

(57) ABSTRACT

There are disclosed compositions comprising guggul and at least one of 1, 3:1, 4-beta-glucan or a beta-sitosterol-containing sterol mixture. The compositions are preferably suitable for reducing cholesterol level. Also disclosed are food and beverage compositions comprising the guggul-containing compositions, that are preferably suitable for reducing cholesterol level. There is also disclosed a method for reducing cholesterol level comprising administering to a human or animal, an effective amount of the guggul-containing compositions, or a food or beverage composition that comprises the guggul-containing compositions.

### FIELD OF THE INVENTION

The present invention relates to compositions suitable for reducing cholesterol levels in humans and animals. The present invention also relates to methods for reducing cholesterol levels in humans and animals.

### BACKGROUND OF THE INVENTION

Guggul is a well known extract from the resin of the mukul myrrh tree (Commiphora mukul). Guggul has been used in the treatment of obesity and lipid disorders. Guggulipid, an ethyl acetate extract of the resin, has been used in the treatment of hyperlipidemia, and contains two compounds, E-guggulsterone and Z-guggulsterone, that decrease hepatic cholesterol levels.

Plant sterols are known to inhibit the absorption and/or reabsorption of cholesterol (external and recycled).

It would be desirable to have products that effectively reduce cholesterol levels in humans and animals, and methods for effectively reducing cholesterol levels in humans and animals.

Accordingly, it is an object of the present invention to provide compositions that are suitable for reducing cholesterol levels in humans and animals.

It is a further object of the present invention to provide food and beverage compositions that are suitable for reducing cholesterol levels in humans and animals.

It is a still further object of the present invention to provide methods for reducing cholesterol levels in humans and animals.

### SUMMARY OF THE INVENTION

The present invention relates to compositions comprising guggul and at least one or more of a beta-sitosterol-containing sterol mixture, and 1, 3:1, 4-beta-glucan.

The present invention also relates to food and beverage compositions comprising a food or beverage and a composition comprising guggul and at least one or more of a beta-sitosterol-containing sterol mixture, and 1, 3:1, 4-beta-glucan.

The present invention also relates to methods for reducing cholesterol levels in humans and animals comprising administering to humans and animals an effective amount of any of a composition comprising guggul and at least one or more of a beta-sitosterol-containing sterol mixture, and 1, 3:1, 4-beta-glucan, or a food or beverage composition comprising a food or beverage and a composition comprising guggul and at least one or more of a beta-sitosterol-containing sterol mixture, and 1, 3:1, 4-beta-glucan.

The compositions of the present invention, and foods and beverages comprising the compositions of the present invention, are expected to effectively reduce the cholesterol levels in humans and animals.

### DETAILED DESCRIPTION OF THE INVENTION

The present invention relates to compositions comprising guggul and at least one or more of a beta-sitosterol-containing sterol mixture, and 1, 3:1, 4-beta-glucan.

The present invention also relates to food and beverage compositions comprising a food or beverage and a composition comprising guggul and at least one or more of a beta-sitosterol-containing sterol mixture, and 1, 3:1, 4-beta-glucan.

The present invention also relates to methods for reducing cholesterol levels in humans and animals comprising administering to humans and animals an effective amount of any of a composition comprising guggul and at least one or more of a beta-sitosterol-containing sterol mixture, and 1, 3:1, 4-beta-glucan, or a food or beverage composition comprising food or beverage and a composition comprising guggul and at least one or more of a beta-sitosterol-containing sterol mixture, and 1, 3:1, 4-beta-glucan.

The compositions of the present invention, and foods and beverages comprising the compositions of the present invention, are expected to effectively reduce the cholesterol levels in humans and animals.

In producing the compositions of the present invention, as mentioned herein, guggul is a well-known, available extract from the resin of the mukul myrrh tree (Commiphora mukul).

The 1, 3:1, 4-beta-glucan used herein is known and available.

Any beta-sitosterol-containing sterol mixture is suitable for use herein. Exemplary beta-sitosterol-containing sterol mixtures include, but are not limited to the following:

| Sterol | Canola | Corn | Soybean | Sunflower | Tall Oil |
|---|---|---|---|---|---|
| Cholesterol | 0.1 | 0.1 | 0.3 | 0.1 | — |
| Brassicasterol | 13.8 | — | — | — | — |
| Campesterol | 27.6 | 17.2 | 18.1 | 7.5 | 14 |
| Stigmasterol | 0.5 | 6.3 | 15.2 | 7.5 | — |
| β-sitosterol | 52.3 | 60.3 | 54.1 | 58.2 | 60 |
| D5-Avenasterol | 1.9 | 10.5 | 2.5 | 4.0 | — |
| D7-Avenasterol | 1.1 | 1.1 | 2.0 | 4.0 | — |
| D7-Stigmasterol | 2.3 | 1.8 | 1.4 | 7.1 | — |
| Campestanol | — | — | — | — | 3 |
| β-Sitostanol | — | — | — | — | 20 |

In producing the compositions comprising guggul and either a beta-sitosterol-containing sterol mixture or 1, 3:1, 4-beta-glucan, the guggul is present in an amount ranging from about 1 to about 99% by weight, based on the composition, and the beta-sitosterol-containing sterol mixture or the 1, 3:1, 4-beta-glucan is present in an amount ranging from about 1 to about 99% by weight, based on the composition.

Preferably, the guggul is present in an amount ranging from about 5 to about 15% by weight of the composition, and most preferably in an amount of about 10% by weight. The beta-sitosterol-containing sterol mixture or 1, 3:1, 4-beta-glucan is preferably present in an amount ranging from about 85 to about 95% by weight, and most preferably, in an amount of about 90% by weight.

In producing the compositions comprising guggul and both a beta-sitosterol-containing sterol mixture and 1, 3:1, 4-beta-glucan, the guggul is present in an amount ranging from about 1 to about 99% by weight based on the composition, the beta-sitosterol-containing sterol mixture is present in an amount ranging from about 1 to about 99% by weight, based on the composition, and the 1, 3:1, 4-beta-glucan is present in an amount ranging from about 1 to about 99% by weight based on the composition.

Preferably, the guggul is present in an amount ranging from about 7 to about 21% by weight, based on the composition, and most preferably, in an amount of about 14% by weight. The beta-sitosterol-containing-sterol mixture is preferably present in an amount ranging from about 40 to about 47% by weight based on the composition, and most preferably, in an amount of about 43% by weight. The 1, 3:1, 4-beta-glucan is preferably present in an amount ranging from about 40 to about 47% by weight based on the composition, and most preferably, in an amount of about 43% by weight.

The compositions comprising guggul and at least one or more of a beta-sitosterol-containing sterol mixture, and 1, 3:1, 4-beta-glucan, may be produced by any conventional technique, such as, for example, by mixing the components of the compositions.

The food and beverage compositions of the present invention comprise a food or a beverage and a composition as described herein comprising guggul and at least one or more of a beta-sitosterol-containing sterol mixture, and 1, 3:1, 4-beta-glucan. In preparing the compositions, any food may be used, or any beverage may be used. Examples of suitable foods include, but are not limited to, margarine, shortening, ice cream, yogurt.

In a more extensive non-limiting description of suitable foods and beverages where the guggul-containing compositions herein may be incorporated, the following may be mentioned:
(a) Fat-based food products including cooking and frying oils, salad dressings, mayonnaise, margarines, table spreads, cheeses (soft and hard), cream cheese, cottage cheese, frozen desserts (ice cream, ice milk, low fat frozen desserts), dips, sauces and creamers.
(b) Beverages including milks, milk alternatives, juices, juice drinks, smoothies, meal replacement beverages, weight loss beverages, protein fortified beverages, sports beverages, tea-based beverages, coffee-based beverages, drinkable yogurts, instant and dry mix beverages.
(c) Confections including confectionery coatings, confectionery chews, caramels, nougat.
(d) Bakery products including breads, muffins, rolls, biscuits, buns, cookies, cake mixes, brownie mixes, bread mixes, granola bars, protein fortified bars and nutritional bars.
(e) Other food applications including cereals, pastas, soups.

The food and beverage compositions herein comprise the food or beverage and an amount of greater than 0 to about 6 grams, preferably about 2 to about 6 grams, and most preferably about 4 grams, of a composition as defined herein comprising guggul and either a beta-sitosterol-containing sterol mixture, or 1, 3:1, 4-beta-glucan. In such compositions, the guggul is present in an amount ranging from about 1 to about 99% by weight based on the composition, more preferably about 5 to about 15% by weight, and most preferably about 10% by weight. The beta-sitosterol-containing sterol mixture or the 1, 3:1, 4-beta-glucan is present in an amount ranging from about 1 to about 99% by weight based on the composition, more preferably about 85 to about 95% by weight, and most preferably, in an amount of about 90% by weight, based on the composition.

The food and beverage compositions herein comprise the food or beverage and an amount of greater than 0 to about 10 grams, preferably about 5 to about 10 grams, and most preferably about 7 grams, of a composition as defined herein comprising guggul and both a beta-sitosterol-containing sterol mixture and 1, 3:1, 4-beta-glucan. In such compositions, the guggul is present in an amount ranging from about 1 to about 99% by weight, based on the composition, more preferably about 7 to about 21% by weight, and most preferably, about 14% by weight. The beta-sitosterol-containing sterol mixture is preferably present in an amount ranging from about 40 to about 47% by weight based on the composition, and most preferably, in an amount of about 43% by weight. The 1, 3:1, 4-beta-glucan is preferably present in an amount ranging from about 40 to about 47% by weight based on the composition, and most preferably, in an amount of about 43% by weight.

The method herein for reducing the cholesterol level of a human or animal comprises administering to a human or animal an effective amount of a composition comprising guggul and at least one or more of a beta-sitosterol-containing sterol mixture, and 1, 3:1, 4-beta-glucan, or a food or beverage composition comprising a composition comprising guggul and at least one or more of a beta-sitosterol-containing sterol mixture, and 1, 3:1, 4-beta-glucan. More particularly, the method of the present invention comprises administering the guggul-containing compositions, and food and beverage compositions comprising the guggul-containing compositions such that there is administered to a human or an animal an amount of from greater than 0 to about 6 grams, preferably about 2 to about 6 grams, and most preferably about 4 grams, of a composition as defined herein comprising guggul and either a beta-sitosterol-containing sterol mixture, or 1, 3:1, 4-beta-glucan. In such compositions, the guggul is present in an amount ranging from about 1 to about 99% by weight based on the composition, more prefereably about 5 to about 15% by weight, and most preferably about 10% by weight. The beta-sitosterol-containing sterol mixture or the 1, 3:1, 4-beta-glucan is present in an amount ranging from about 1 to about 99% by weight based on the composition, more preferably about 85 to about 95% by weight, and most preferably, in an amount of about 90% by weight, based on the composition.

When the guggul composition being administered comprises guggul and both a beta-sitosterol-containing sterol mixture and 1, 3:1, 4-beta-glucan, there is administered, whether or not incorporated in a food or beverage composition, an amount of greater than 0 to about 10 grams, preferably about 5 to about 10 grams, and most preferably about 7 grams of the guggul-containing composition. In such compositions, the guggul is present in an amount ranging from about 1 to about 99% by weight, based on the composition, more preferably about 7 to about 21% by weight, and most preferably about 14% by weight. The beta-sitosterol-containing sterol mixture is preferably present in an amount ranging from about 40 to about 47% by weight based on the composition, and most preferably, in an amount of about 43% by weight. The 1, 3:1, 4-beta-glucan is preferably present in an amount ranging from about 40 to about 47% by weight based on the composition, and most preferably, in an amount of about 43% by weight.

The administering of the guggul-containing compositions as described herein may be carried out in any known manner, such as, for example, by orally administering the compositions, whether or not incorporated in a food or beverage, to an animal or human.

The invention will be more readily understood by reference to the following examples. There are, of course, many other forms of this invention which will become obvious to one skilled in the art, once the invention has been fully disclosed, and it will accordingly be recognized that these examples are given for the purpose of illustration only, and are not to be construed as limiting the scope of this invention in any way.

### EXAMPLE 1

In this example, 10 weight % guggul and 90 weight % 1, 3:1, 4-beta -glucan are mixed to provide a composition, that is administered to a human. It is expected that the cholesterol level will be effectively reduced.

### EXAMPLE 2

In this example, 5 weight % guggul and 95 weight % canola oil are mixed to provide a composition, that is administered to a human. It is expected that the cholesterol level will be effectively reduced.

### EXAMPLE 3

In this example, 14 weight % guggul, 43 weight % 1, 3:1, 4-beta-glucan and 43 weight % corn oil are mixed to provide a composition, that is administered to a human. It is expected that the cholesterol level will be effectively reduced.

### EXAMPLE 4

In this example, a composition comprising 15 weight % guggul and 85 weight % 1, 3:1, 4-beta-glucan is prepared by mixing the components. The resulting guggul-containing composition is then mixed with ice cream to provide a food composition, that is administered to a human. It is expected that the cholesterol level will be effectively reduced.

### EXAMPLE 5

In this example, a composition comprising 25 weight % guggul and 75 weight % soybean oil is prepared by mixing the components. The resulting guggul-containing composition is then mixed with milk to provide a beverage composition, that is administered to a human. It is expected that the cholesterol level will be effectively reduced.

### EXAMPLE 6

In this example, 7 weight % guggul, 47 weight % 1, 3:1, 4-beta-glucan, and 47 weight % sunflower oil are mixed to provide a composition. The resulting guggul-containing composition is then mixed with caramels to provide a food composition that is administered to a human. It is expected that the cholesterol level will be effectively reduced.

The invention has been described with reference to various specific and illustrative embodiments and techniques. However, one skilled in the art will recognize that many variations and modifications may be made while remaining within the spirit and scope of the invention.

1. A composition comprising guggul and at least one or more components selected from the group consisting of 1, 3:1, 4-beta-glucan and a beta-sitosterol-containing sterol mixture, wherein the guggul is present in an amount ranging from about 1% to about 99% by weight, based on the composition, and the at least one or more components is present in an amount ranging from about 1% to about 99% by weight, based on the composition.

2. The composition according to claim 1 wherein the component is 1, 3:1, 4-beta-glucan.

3. The composition according to claim 1 wherein the component is a beta-sitosterol-containing sterol mixture.

4. The composition according to claim 2 wherein the guggul is present in an amount ranging from about 5% to about 15% by weight, and the 1, 3:1, 4-beta-glucan is present in an amount ranging from about 85% to about 95% by weight.

5. The composition according to claim 3 wherein the guggul is present in an amount ranging from about 5% to about 15% by weight, and the beta-sitosterol-containing sterol mixture is present in an amount ranging from about 85% to about 95% by weight.

6. The composition according to claim 1 comprising guggul, 1, 3:1, 4-beta-glucan and a beta-sitosterol-containing sterol mixture, wherein the guggul is present in an amount ranging from about 7% to about 21% by weight, the 1, 3:1, 4-beta-glucan is present in an amount ranging from about 40% to about 47% by weight, and the beta-sitosterol-containing sterol mixture is present in an amount ranging from about 40% to about 47% by weight.

7. The composition according to claim 1 wherein the guggul is present in an amount of about 10% by weight, and the at least one or more components is present in an amount of about 90% by weight.

8. The composition according to claim 6 wherein the guggul is present in an amount of about 14% by weight, the 1, 3:1, 4-beta-glucan is present in an amount of about 43% by weight, and the beta-sitosterol-containing sterol mixture is present in an amount of about 43% by weight.

9. A food composition comprising a food and a second composition according to claim 1.

10. The food composition according to claim 9 wherein the second composition comprises guggul and one component selected from the group consisting of 1, 3:1, 4-beta-glucan and a beta-sitosterol-containing sterol mixture.

11. The food composition according to claim 10 wherein the second composition is present in an amount ranging from greater than 0 to about 6 grams.

12. The food composition according to claim 11 wherein the second composition is present in an amount ranging from about 2 to about 6 grams.

13. The food composition according to claim 9 wherein the second composition comprises guggul, 1, 3:1, 4-beta-glucan, and a beta-sitosterol-containing sterol mixture.

14. The food composition according to claim 13 wherein the second composition is present in an amount ranging from greater than 0 to about 10 grams.

15. The food composition according to claim 14 wherein the second composition is present in an amount ranging from about 5 to about 10 grams.

16. A beverage composition comprising a beverage and a second composition according to claim 1.

17. The beverage composition according to claim 16 wherein the second composition comprises guggul and one component selected from the group consisting of 1, 3:1, 4-beta-glucan, and a beta-sitosterol-containing sterol mixture.

18. The beverage composition according to claim 17 wherein the second composition is present in an amount ranging from greater than 0 to about 6 grams.

19. The beverage composition according to claim 18 wherein the second composition is present in an amount ranging from about 2 to about 6 grams.

20. The beverage composition according to claim 16 wherein the second composition comprises guggul, 1, 3:1, 4-beta-glucan, and a beta-sitosterol-containing sterol mixture.

21. The beverage composition according to claim 20 wherein the second composition is present in an amount ranging from greater than 0 to about 10 grams.

22. The beverage composition according to claim 21 wherein the second composition is present in an amount ranging from about 5 to about 10 grams.

23. A method for reducing cholesterol level in a human or animal comprising administering to at least one of the human or animal an amount effective to decrease cholesterol levels in the body of said animal or human, of a composition comprising guggul and at least one or more components selected from the group consisting of 1, 3:1, 4-beta-glucan and a beta-sitosterol-containing sterol mixture.

24. The method according to claim 23 wherein the composition comprises guggul and one component selected from the group consisting of 1, 3:1, 4-beta-glucan and a beta-sitosterol-containing sterol mixture.

25. The method according to claim 24 wherein the composition is administered in an amount ranging from greater than 0 to about 6 grams.

26. The method according to claim 25 wherein the composition is administered in an amount ranging from about 2 to about 6 grams.

27. The method according to claim 23 wherein the composition comprises guggul, 1, 3:1, 4-beta-glucan, and a beta-sitosterol-containing sterol mixture.

28. The method according to claim 27 wherein the composition is administered in an amount ranging from greater than 0 to about 10 grams.

29. The method according to claim 28 wherein the composition is administered in an amount ranging from about 5 to about 10 grams.

30. A method for reducing cholesterol level in a human or animal comprising administering to at least one of the human or animal an amount effective to decrease cholesterol level in the body of said animal or human of a food composition comprising a food and a second composition according to claim 1.

31. The method according to claim 30 wherein the second composition comprises guggul and one component selected from the group consisting of 1, 3:1, 4-beta-glucan and a beta-sitosterol-containing sterol mixture.

32. The method according to claim 31 wherein the second composition is administered in an amount ranging from greater than 0 to about 6 grams.

33. The method according to claim 32 wherein the second composition is administered in an amount ranging from about 2 to about 6 grams.

34. The method according to claim 30 wherein the second composition comprises guggul, 1, 3:1, 4-beta-glucan, and a beta-sitosterol-containing sterol mixture.

35. The method according to claim 34 wherein the second composition is administered in an amount ranging from greater than 0 to about 10 grams.

36. The method according to claim 35 wherein the second composition is administered in an amount ranging from about 5 to about 10 grams.

37. A method for reducing cholesterol level in a human or animal comprising administering to at least one of the human or animal an amount effective to decrease cholesterol level in the body of said animal or human of a beverage composition comprising a beverage and a second composition according to claim 1.

38. The method according to claim 37 wherein the second composition comprises guggul and one component selected from the group consisting of 1, 3:1, 4-beta-glucan and a beta-sitosterol-containing sterol mixture.

39. The method according to claim 38 wherein the second composition is administered in an amount ranging from greater than 0 to about 6 grams.

40. The method according to claim 39 wherein the second composition is administered in an amount ranging from about 2 to about 6 grams.

41. The method according to claim 37 wherein the second composition comprises guggul, 1, 3:1, 4-beta-glucan, and a beta-sitosterol-containing sterol mixture.

42. The method according to claim 41 wherein the second composition is administered in an amount ranging from greater than 0 to about 10 grams.

43. The method according to claim 42 wherein the second composition is administered in an amount ranging from about 5 to about 10 grams.
